(19) United States
(12) Patent Application Publication
(10) Pub. No.: US 2004/0156833 A1
(43) Pub. Date: Aug. 12, 2004

(54) Devices and methods for implanting transduced cells

(75) Inventors: William R. A. Osborne, Seattle, WA (US); Randolph L. Geary, Seattle, WA (US); Stella Lau, Seattle, WA (US); Alexander W. Clowes, Seattle, WA (US); David C. Dale, Seattle, WA (US)

Correspondence Address: TOWNSEND and TOWNSEND and CREW, Two Embarcadero Center, 8th Floor, San Francisco, CA, 94111, US

(73) Assignee: University of Washington

(21) Appl. No.: 10/765,306

(22) Filed: Jan. 26, 2004

Related U.S. Application Data
(63) Continuation of application No. 08/217,324, filed on Mar. 24, 1994, now abandoned.

(51) Int. Cl. A61K 48/00

(52) U.S. Cl. 424/93.21

(57) ABSTRACT

Vascular grafts are used in gene therapy to implant autologous vascular smooth muscle cells that have been transduced with the gene of interest. The smooth muscle cells are immobilized within the pores and upon the interior surface of the wall of the graft, which is then coated with vascular endothelial cells and implanted into a patients's vascular system. The grafting device can be used in methods of treatment, such as supplying erythropoietin to a patient, and can be removed easily and replenished as necessary. The process is readily controlled and significantly reduces the risk of a viral gene vector spreading beyond a target cell population.

### GOVERNMENT SUPPORT

This invention was made with U.S. government support under grants from the National Institutes of Health. The U.S. government may have certain rights in this invention.

### BACKGROUND OF THE INVENTION

Gene therapy presents unparalleled opportunities to treat and prevent human disease. Gene therapy involves the genetic alteration of an individual's cells to provide a gene product that will ameliorate, cure or prevent a disease. It has been proposed to treat a wide variety of genetic and acquired conditions associated with defective or missing proteins such as enzymes, cytokines, hormones, cellular adhesion molecules and other receptors, and the like. Clinical trials are under way to modify an individual's immune system to treat specific cancers, and trials have been proposed to use gene therapy to treat certain infectious diseases.

With the goal of applying gene therapy to a broad spectrum of human diseases, a variety of cell types have been proposed as targets for inserting the gene(s) of interest. These include, for example, hematopoietic cells., fibroblasts, lymphocytes, keratinocytes, hepatocytes, muscle cells, and endothelial cells. Miller, Blood 76: 271-278 (1990). Difficulties in obtaining efficient gene transfer and persistent gene expression in many of these cells, however, have limited the application of gene therapy techniques. For example, while hematopoietic cells are often a primary target of gene therapy, experiments with gene therapy using hematopoietic cells in larger animals have generally resulted in transient presence of gene function or low levels of expression, even though more successful gene transfer and long-term expression has been achieved in rodents. Miller, Nature 357: 455-460 (1992). Similarly, injection of DNA directly into skeletal muscle of non-human primates has resulted in low levels of gene expression, and attempts to transfer genes into other tissues using DNA have not been successful. Another technique involves the injection of genetically modified myoblasts or fibroblasts into skeletal muscle, but these approaches carry a risk of possible tumor development (depending on the origin of the injected cell) and suppression of the transferred gene.

In addition to skeletal muscle myoblasts, smooth muscle cells have been explored as a possible vehicle for gene transfer. Lynch et al., Proc. Natl. Acad. Sci. USA 89: 1138-1142 (1992); Plautz et al., Circulation 83: 578-583 (1991); Nabel et al., Science 249: 1285-1288 (1990). Smooth muscle cells are the predominant cell type within the vasculature and exist as a multilayered mass of long-lived cells in proximity to circulating blood. Smooth muscle cells play a critical role in arterial growth and development as well as in vascular diseases. Smooth muscle cells in blood vessels can be removed, cultured, and genetically modified. The cells can then be returned to blood vessels by disrupting the vessel wall. Long term vector-encoded gene expression from retrovirally transduced smooth muscle cells has been reported in rodents (Lynch et al., supra). Autologous rat smooth muscle cells transduced with human genes in vitro were seeded back into rat carotid arteries that were denuded of endothelium.

In man, however, it is quite unlikely that seeding of cultured cells into denuded or otherwise disrupted arteries will be feasible. Therefore other strategies are needed to deliver genes of interest for local or systemic effects. Desirably, the methods should employ a well characterized and relatively homogeneous population of autologous target cells. The genetic manipulations of the cell population are preferably performed outside of the body to better control the process as well as to minimize the risk of the viral vector spreading beyond the target cells. In contrast, the site-specific transformation of cells described in Nabel et al., PCT publication WO 90/11734, involves an in vivo infection of targeted cells and exposes a relatively undefined cell population, such as endothelial cells, smooth muscle cells, macrophages and fibroblasts, to the viral vector and the risks attendant therewith.

New strategies for delivering genes of interest to patients should also provide a clinician with the capability to more precisely regulate the amount of gene product as may be necessary for a particular patient or disease. The procedures should further allow for convenient and periodic replenishment, or even complete replacement, of the cells which express the gene, as expression may sometimes be limited in duration or amount or other situations may necessitate removal of the genetically modified cells. In some previous methods, such as described in Nabel, supra, modified cells are free to migrate beyond the area in which they are instilled, thus making complete removal from the patient a difficult if not impossible task. Quite surprisingly, the present invention fulfills these and other related needs.

### SUMMARY OF THE INVENTION

The present invention provides methods and devices for treatment of a genetic or acquired disease in a host by gene therapy. In general terms, the methods for introducing one or more genes into a patient comprise engrafting a device into the patient's vascular system, where the device contains immobilized transduced cells which contain the gene of interest. The transduced cells are smooth muscle cells, typically vascular smooth muscle cells which have been obtained from the individual in whom the device is to be engrafted. The transduced smooth muscle cells are intercalated within the pores and are adherent to the interior surface of the graft. The graft is conveniently a synthetic vascular prostheses of a porous flexible material that is longate and tubular in design and suitable for use as a vascular graft. Autologous vascular endothelial cells are used to coat the interior surface of graft containing the transduced smooth muscle cell complex. The device is then implanted into the vasculature of the individual and the transduced gene expresses the product of interest. The expressed product of interest can be secreted into the surrounding tissues and fluids of the circulatory system of the patient or not secreted.

Typically the porous synthetic material used to form the basis of the graft is polytetrafluoroethylene (PTFE), Dacron, polyurethane, Corethane®, nylon or various composites thereof. The pores of the graft will usually be about 60 to 90 microns in diameter, and the graft material itself may be wrapped or unwrapped. The internal diameter of the graft will be of a size appropriate to the vessel in which it is to be implanted. The length of the graft and the number of transduced smooth muscle cells seeded therein will contribute to regulating the amount of gene product that is expressed in the host.

In another embodiment the method comprises first removing a segment of vascular tissue from an individual in need of gene therapy. The endothelial cells and smooth muscle cells are removed from the vascular tissue and cultivated. The smooth muscle cells are then transduced with one or more genes which encode the product(s) of interest. Typically the gene will be part of a vector designed for expression of the gene and a selectable marker, such as a retroviral vector. The gene can encode any of a variety of products, such as enzymes, cytokines, receptors, hormones, growth factors, coagulation factors, and the like. The transduced smooth muscle cells are then selected for by the selectable marker, and then amplified in culture. The amplified transduced smooth muscle cells are then harvested and suspended in medium containing autologous serum and, optionally, collagen or other proteins facilitating cell adhesion and/or proliferation, and then introduced into the graft, typically by filtration. The transduced smooth muscle cells are allowed to become adherent to the graft complex and intercalate into the interstices thereof, and then coated with a preparation of the vascular endothelial cells, which are optionally suspended in medium containing autologous serum. The device of endothelial cell/transduced smooth muscle cell/graft is allowed to incubate in medium containing autologous serum from 30 min. to five or more days, and then implanted into the patient's circulatory system. In a method for treating an occlusion of a blood vessel in a patient, the transduced cells will constitutively express an anticoagulant and the device is engrafted into the occluded blood vessel, bypassing the occlusion.

### DESCRIPTION OF THE SPECIFIC EMBODIMENTS

The present invention provides methods and devices to treat individuals via gene therapy. By virtue of grafts containing transduced autologous cells which express the gene of interest, the gene product is supplied to the treated individual. The graft containing the transduced, gene-expressing cells can be removed and replaced if necessary. Additionally, by adjusting the size of the graft and/or the density of cells intercalated therein, the amount of gene product can be regulated. The grafts containing the transduced cells can be used to provide local or systemic gene therapy in patients. Because the grafts containing the genetically modified cells can be placed in close proximity to the circulation, this procedure can be applied to gene therapy involving both secreted and non-secreted proteins.

The device for implanting the transduced cells in a patient typically comprises a porous material suitable for implantation in th vascular system of a patient. The device will preferably be a tubular elongate member having a wall, which wall has an interior surface, an exterior surface, and pores therein. The material should be relatively non-immmunogenic and non-thrombogenic, and sufficiently porous to allow transudation of proteins and the like from the vascular circulation, but not complete cells. The pores should allow smooth muscle cells to intercalate in the interstices of the graft, e.g., about 40 to 100 microns, more usually about 60 to 90 microns.

The material which comprises the grafting device will generally be a porous member of the teflon family which is suitable for surgical implantation in the vascular system of a patient. Preferably the material is expanded polytetrafluoroethylene, PTFE (W.L. Gore & Associates, Inc. Flagstaff, Ariz., or IMPRA). Other suitable materials include polyesters (e.g., Dacron®; Vascutek Ltd., Paisley, Glasgow, Scotland), polyurethane, Corethane®, nylon and various composites thereof (e.g., as described in Stanley et al., Vascular Surgery W. S. Moore, ed., Grune & Stratton, Orlando, Fla., 1986, pp. 365-388, and Wilson et al., Science 244:1344-1346 (1989)). The graft material will be wrapped or unwrapped, but must be able to withstand pressures associated with the vascular system and a limited amount of swelling. The internal diameter of the graft can vary widely depending on the location of the implant and other factors, but will generally be about 2 to 8 mm, more preferably about 4 to 6 mm. In some embodiments the graft can even be a segment of vein obtained from the patient.

According to the present invention, vascular endothelial cells and smooth muscle cells are removed from an individual for seeding into the vascular graft of the invention. The smooth muscle cells are transduced with a gene encoding the protein of interest and together with the endothelial cells are seeded into the grafting device and subsequently implanted into the individual from which the cells were removed, i.e., implants of transduced autologous cells.

The vascular endothelial cells and smooth muscle cells need not be from the same blood vessel, although most conveniently they are isolated from the same segment of vascular tissue that is removed from the patient. The vascular tissue can be removed from any of several sites, but should be from a site that causes the least amount of discomfort and potential harm to the patient, e.g., superficial veins such as the lesser saphenous vein, greater saphenous vein, cephalic vein, and other similar veins. The amount of vessel which is removed should be sufficient to supply endothelial cells for propagation and smooth muscle cells for transduction of the gene of interest at a low passage number, e.g., typically at least approximately 2 cm and up to 10 cm or more in length, although this may vary widely depending on the vessel, the condition of the patient and the disease to be treated by the gene therapy.

Endothelial cells are selectively removed from the excised blood vessel or other micro- or macrovascular tissue according to techniques known to those in the art. See, e.g., Vohra et al., Vasc. Surg. 22: 393-397 (1989); Folkman et al., Proc. Natl. Acad. Sci. USA 76: 5217-5221 (1979); Kern et al., J. Clin. Invest. 71: 1822-1829 (1983) and Sterpetti et al., J. Surg. Res. 48:101-106 (1990), which are incorporated herein by reference. For example, a solution of Dispase can be introduced into a excised vessel, which is then incubated for a short period and the cells then flushed from the vein. Endothelial cells are then collected and can be expanded by cultivation in an appropriate culture medium, e.g., on gelatin-coated dishes in RPMI 1640, 20% FBS containing heparin and endothelial cell growth supplement under standard conditions (e.g, humidified incubator with 5% CO₂/95% air at 37° C.). Endothelial cells are obtained in a number adequate for luminal seeding of grafts. The number of endothelial cells can be calculated based on surface area and assuming about a 10-20% seeding efficiency. Thus, for a 10 cm graft of 0.2 cm radius, with a surface area of 12 cm², endothelial cells were pooled from two nearly confluent Petrie dishes (a 10 cm diameter Petrie dish having a surface area of 75 cm²) to yield about 4×10⁶ cells for the seeding procedure.

After th endothelial cells are harvest d from the excised vessel the smooth muscle cells are removed using establish d techniques, e.g., enzyme digestion or outgrowth from cultured pieces of vessel. For enzyme digests the de-endothelialized vessel segments are stripped of adventitia and treated with an appropriate enzyme solution, e.g., collagenase P and elastase in a solution of serum albumin. To isolate smooth muscle cells from cultured explants, small pieces of de-endothelialized vessel (e.g., 2 mm²) are plated in an appropriate culture solution, e.g., Dulbecco's modified Eagle's medium and 10% serum and incubated at 37° C. at 5% CO₂. When outgrowth of smooth muscle cells is attained tissue fragments are removed and the smooth muscle cells expanded. Cell types are confirmed by immunocytochemical staining.

The smooth muscle cells are transduced with at least one gene encoding the expression product of interest, typically a protein. The protein is one which confers a benefit to the patient, either directly or indirectly. The gene may encode a secreted or non-secreted protein, or an active portion thereof. The selection of a suitable gene for the condition being treated will be apparent to those skilled in the art. By “gene” is meant DNA that encodes a desired product, such as, for example, a cytokine, a clotting factor, a hormone, an enzyme, a transport protein, a regulatory protein, a structural protein, a receptor, an antigen, etc. Representative examples of genes for introducing into humans are those encoding human erythropoietin (described in U.S. Pat. No. 4,703,008), human G-CSF, human GM-CSF (Anderson et al., Proc. Natl. Acad. Sci. USA 82:6250 (1985)), plasminogen activator, urokinase, insulin (e.g., human insulin as described in U.S. Pat. No. 4,652,525 or proinsulin described in U.S. Pat. No. 4,431,740), interleukins (e.g., interleukin-1, interleukin-2 [described in U.S. Pat. No. 4,738,927], interleukin-3 [described in EP Publ. 275,598 and 282,185], interleukin-4, interleukin-7 [U.S. Pat. No. 4,965,195], etc.), interferons, Factor VIII, Factor IX, von Willebrand Factor, ADA, human growth hormone (described in U.S. Pat. No. 4,342,832), etc., analogs and fusions thereof (e.g., fusions of GM-CSF and IL-3 [U.S. Pat. No. 5,108,910]. Each of the foregoing patents and publications is expressly incorporated herein by reference.

It is possible and may be desirable in some instances to employ a mixture of smooth muscle cells which include a first group transduced with a gene of interest and a second group transduced with a second, different gene of interest. Alternatively, the smooth muscle cells may be transduced with more than one gene of interest.

The genes are transduced into the smooth muscle cells using well established protocols. Typically the gene transfer vector will be a retroviral vector, but other vectors may also be employed, e.g., adenovirus vectors (e.g., Rosenfeld et al., Cell 68: 143-155 (1992) and Curiel et al., Proc. Natl. Acad. Sci. USA 88: 8850-8854 (1991), adenovirus associated vectors (e.g., Muzyczka, Curr. Top. Microbiol. Immunol. 158: 97-129 (1992), and as reviewed by Miller, Nature 357: 455-460 (1992), which publications are incorporated herein by reference). The construction of retroviral vectors has been described, e.g., Miller and Rosman, Biotechniques 7: 980-990 (1989); Adam et al., J. Virol. 65: 4985-4990 (1991); Miller, Curr. Top. Microbiol. Immunol. 158: 1-24 (1992); UK Patent publication GB 2,269,175A; and pending U.S. patent application Ser. No. 07/743,513, each of which is incorporated herein by reference. A preferred retroviral vector is made using PA317 amphotropic retrovirus packaging cells, as described in Miller and Buttimore, Molec. Cell. Biol. 6: 2895-2902 (1986), also incorporated herein by reference.

Transduced smooth muscle cells containing the desired gene(s) of interest are cultured, typically in the presence of a selection agent, e.g., G418, neomycin or the like depending on the selectable marker used in the vector, and then may be expanded in the absence of the selection agent until a sufficient number of cells are available for graft seeding. Prior to seeding the graft the transduced smooth muscle cells are harvested and placed in medium (e.g., DMEM) containing heat-inactivated autologous serum and, optionally, type I collagen or other protein such as fibronectin may be added to the suspension. The concentration of autologous serum can vary considerably, e.g., from about 5% up to 60% or more, but typically is about 10% to 50%, more usually about 20% to about 40%.

The suspension of transduced smooth muscle cells and media is then introduced into the graft device using sterile technique. Typically one end of the graft is occluded and the transduced smooth muscle cell suspension is introduced into the other and filtered gently through the graft wall. The filtrate may be collected and refiltered through the graft several times. If collagen is included in the suspension the graft is incubated to polymerize the collagen, e.g., about 37° C. for 45 min. Following seeding of the transduced smooth muscle cells and prior to seeding with the endothelial cells the graft may optionally be incubated in media, preferably containing heat-inactivated autologous serum, to improve the integration of the transduced cells into the interstices of the graft. The incubation period may be up to five days or longer.

Endothelial cells are harvested and suspended in medium optionally containing the heat-inactivated autologous serum and are then introduced into the lumen of the smooth muscle cell-seeded grafts. Although a variety techniques can be used to seed the endothelial cell layer, in one embodiment described herein the ends of the graft containing the suspension of endothelial cells is clamped and the graft is rolled 180° at 10 min. intervals over 40 min. to distribute the cells evenly onto the luminal surface. The seeded graft can then be implanted into the patient or can be incubated for an additional period to better anchor the endothelial cell layer and thus increase its resistance to the shear forces it will be subjected to when implanted in the patient's circulation.

In an alternative embodiment the graft can be a segment of vein obtained from the patient. The vein segment is denuded of endothelial by enzyme digestion or mechanical means, as described above, and then seeded with transduced autologous smooth muscle cells that have been obtained previously from the patient and transduced with the gene(s) of interest. The seeded vein segment is then implanted back into the patient's vascular system.

The grafts containing the seeded transduced smooth muscle cells and the endothelial cells are implanted into the patient's vascular system. Typically the graft will communicate with the arterial system, such as, e.g., an arteriovenous fistula as used for an gioaccess and hemodialysis, as arterial bypass grafts, and the like. The graft may be anastomosed to the axillary artery, brachial artery, radial artery, femoral artery, popliteal artery, saphenous vein, basilic vein, cephalic vein, femoral vein, or the like. The graft may be placed by well known methods such as standard end-to-side anastomosis, etc. The graft will be placed in vessels of appropriate size and location as determined by the physician in accordance with the disease being treated by gene therapy and the general condition of the patient. For example, in some instances it may be desirable to placed the seeded graft into the circulation of a patient and bypass the native circulation which is then ligated. In a patient with an occluded blood vessel, the transduced cells of the seeded graft can constitutively express an anticoagulant protein such as plasminogen activator, e.g., alteplase or urokinase, or antithrombin-III. The seeded graft can be placed into the occluded blood vessel, bypassing the occlusion. In a patient with diabetes or susceptible to diabetes, the gene will typically encode human insulin or proinsulin polypeptides.

Once implanted, the graft should be monitored for expression of the gene of interest. A number of assays are known to those in the art for the particular gene product(s) provided to the patient. Depending on whether an inducible promoter control the expression of the gene(s), it may be necessary to provide an agent to the patient that directly or indirectly induces the expression of the gene of interest.

Depending on the vector used and other variables the implanted graft is capable of producing the desired gene product for a considerable period of time. If expression of the gene product diminishes over a period of time, the graft can be removed and replaced with a freshly seeded graft. Thus, the present devices and methods are capable of providing a therapeutic or prophylactic gene product as long as desired, including for the life of the patient. The invention is particularly useful in treatment of anemia or diabetes, which often require long term maintenance therapies.

The present invention makes possible the treatment or prevention of a variety of diseases for which gene therapy is feasible. The patient will be one susceptible or predisposed to a disease condition, or will already be suffering from the disease, even though it may not be diagnosed in the patient. By “patient” is meant to refer to any mammal susceptible to treatment in which engraftment of a vascular device is feasible, but will typically be a human. A variety of veterinary uses are also feasible in accordance with the present invention, and thus engraftment of the device containing the transduced smooth muscle cells in equine, bovine, canine, feline, porcine, and other non-human primate patients can be employed for veterinary conditions susceptible to treatment by gene therapy.

The following examples are offered by way of illustration, not by way of limitation.

### EXAMPLE I

Prosthetic vascular grafts containing retrovirally-transduced autologous vascular smooth muscle cells were used to introduce human genes into non-human primates.

Endothelial cells and smooth muscle cells were both obtained following a single vein biopsy from each baboon. Under general anesthesia an 8 cm length of lesser saphenous vein was excised, side branches were ligated and the ends cannulated to facilitate flushing. After rinsing th lumen with 5 ml of Hank's balanced salt solution ((HBSS), Gibco, New York, N.Y.), 3 ml of a Dispase solution was introduced (2.4 U/ml in HBSS, Boehringer Mannheim Inc., Indianapolis, Ind.) to selectively remove the endothelial cells. The vein was slightly distended with the enzyme solution by occluding venous outflow and then incubated at 37° C. for 20 minutes. Released endothelial cells were gently flushed from the vein lumen with 10 ml HBSS, spun and immediately plated onto gelatin-coated dishes and cultured in RPMI 1640, 20% FBS (vol/vol) containing 90 μg/ml heparin sodium and 100 μg/ml endothelial cell growth supplement (Collaborative Research, Bedford, Mass.) in a humidified incubator with 5% CO₂/95% air at 37° C. Endothelial cells were expanded to a number adequate for lumenal seeding of PTFE grafts (surface area 12.6 cm²/10 cm graft length), or approximately 3×10⁵ cells.

After endothelial cell harvest, smooth muscle cells were removed from the same vein either by enzyme digestion or outgrowth from cultured pieces of vein. For digests, de-endothelialized vein segments were stripped of adventitia and treated with collagenase P (1 mg/ml; Boehringer-Mannheim), elastase (2 mg/ml; Boehringer-Mannheim), soybean trypsin inhibitor (400 μg/ml; Worthington) and bovine serum albumin (1 mg/ml; Sigma). Explants were prepared by plating 2 mm² pieces of de-endothelialized vein in Dulbecco modified Eagle's medium, 10% FBS (vol/vol), incubated at 37° C., 5% CO₂. When outgrowth of smooth muscle cells was obtained, tissue fragments were removed and the smooth muscle cells expanded.

Cultured cells were characterized immunocytochemically with cell specific primary antibodies localized with fluorescein-conjugated secondary antibodies. Smooth muscle cells stained positive with antibodies against alpha-actin (1:100 dilution; Boehringer Mannheim, Indianapolis, Ind.) and vimentin (1:25, Dako; Carpenteria, Calif.) but negative for the endothelial cell marker, factor VIII-related antigen (1:100; Dako). Endothelial cells stained in a reciprocal fashion. Pure cultures of endothelial cells (as determined by immunofluorescence of Factor VIII-associated antigen) and smooth muscle cells (reactivity with antibodies to alpha-actin) were obtained from each animal, as documented by the cell-specific immunofluorescence.

Retroviral vectors encoding the reporter gene E. coli β-galactosidas (β-Gal) or a control gene, human purine nucleoside phosphorylase (PNP), and the selectable neomycin phosphotransferase (neo) gene were constructed as previously described. Lynch et al., Proc. Natl. Acad. Sci. USA 89: 1138-1142 (1992); Osborne and Miller, Proc. Natl. Acad. Sci. USA 85: 6851-6855 (1988); Miller and Rosman, BioTechniques 7:980-990 (1989); and Miller and Buttimore, Mol. Cell. Biol. 6: 2895-2902 (1986), which are incorporated herein by reference. Nomenclature of the vectors (LNPoZ and LPNSN respectively) is based on the order of genetic elements: L, LTR promoter; N, neo; Po, poliovirus IRES 5′ sequences; Z, lacz (β-Gal) gene; PN, PNP; S, simian virus 40 promoter. Amphotropic retroviral vectors were produced from PA 317 packing cells (Miller and Buttimore, supra). LNPoZ producer cells were titered at 5×10⁵ colony forming units (cfu)/ml (Adam et al., J. Virol. 65: 4985-4990 (1991)) and the LPNSN-2 producer line at 1-2×10⁶ cfu/ml (Osborne and Miller, supra).

Early passage (P1 or P2) smooth muscle cells were exposed to viral harvests from the murine amphotropic virus-producing cell lines, PA317/LNPoZ and PA317/LPNSN-2, for a period of 24 h in the presence of Polybrene (4 μg/ml, Sigma). Infected cells were selected in G-418 (1 mg/ml; Gibco) for 10-14 days and then expanded in the absence of G418 for seeding. When at least 5×10⁷ of both LNPoZ and LPNSN-transduced smooth muscle cells were available from an individual baboon the cells were prepared for graft seeding.

Vector expression was monitored from LNPoZ transduced cells by enzyme specific staining. β-galactosidase expression was documented in transduced smooth muscle cell cultures after selection in neomycin phosphotransferase. Virtually all cells stained intensely blue with the X-Gal chromogen. LPNSN-2 expression was assayed in cultured smooth muscle cells by histochemical staining after enzyme separation by starch gel lectrophoresis which clearly distinguished human from endogenous baboon PNP. Extracts of LNPoZ and LPNSN-2 transduced smooth muscle cell had PNP activities of 1.1 μmol/hr/mg protein and 9.0 μmol/hr/mg protein respectively, which represented an 8-fold increase in PNP activity in PNP-vector infected cells.

Within 4 to 5 weeks of obtaining vein biopsies, 10⁷ to 10⁹ transduced smooth muscle cells were available for graft seeding. Endothelial cells (nontransduced) were available more quickly and were stored under liquid nitrogen until needed for seeding. Trypan blue exclusion assays showed greater than 95% viability of both cell types after completion of the seeding process.

Prior to seeding, transduced smooth muscle cells were harvested, counted and placed into 5 ml DMEM containing heat-inactivated (56° C. for 30 min) autologous serum (25% vol/vol) and 0.75 mg/ml type-I cbllagen (Vitrogen 100, Celltrix, Calif.). The smooth muscle cell-collagen suspension was kept cool until used. Using sterile technique, reinforced porous PTFE grafts (W.L. Gore and Associates, Inc., Flagstaff, Ariz.) of 4 mm internal diameter and 10 cm in length were immersed in 95% ethanol and then flushed three times with 5 ml phosphate buffered saline. One end of the wetted graft was occluded and the Smooth muscle cell-collagen suspension was introduced from the other end with a 5 ml syringe and filtered gently through the graft wall. The filtrate was collected and refiltered a total of four to six times. Grafts were then unclamped, drained and warmed at 37° C. for up to 45 minutes until the collagen polymerized. Grafts were then covered with media and incubated overnight. The following morning, endothelial cell cultures were harvested, suspended in 2 ml media with 10% heat-inactivated autologous serum. The endothelial cell suspension was used to seed the lumenal surface of smooth muscle cell-seeded grafts. The endothelial cell suspension was introduced into the graft and the ends clamped. Grafts were rolled 180° at 10 minute intervals over 40 minutes to distribute the cells evenly. Seeded grafts were then delivered to the surgical suite for immediate implantation. Microscopic analysis of seeded graft cross-sections immediately prior to implantation showed smooth muscle cells distributed throughout th graft wall. In the cross-sections, a graft seeded with β-galactosidase-expressing smooth muscle cells prior to implantation showed many cells staining blue with the X-Gal chromogen, and were seen distributed throughout the graft wall. LPNSN-2 transduced cells did not stain with the X-Gal chromogen.

Seeded grafts (one graft seeded with LNPoZ and one with LPNSN) were placed into the aorto-iliac circulation of 4 young male baboons (Papio cynocephalus) weighing ˜10 kg. Anesthesia was induced with intramuscular ketamine hydrochloride (10 mg/kg) and maintained with inhaled halothane. Antibiotics were administered intramuscularly (cefazolin sodium, 25 mg/kg, Bristol-Myers Squibb, Princeton, N.J.) and heparin sodium administered intravenously (200 U/kg, Elkins-Sinn, Inc., Cherry Hill, N.J.). Grafts were sutured end-to-side to the infrarenal aorta and to the common iliac artery bilaterally using 6-0 polypropylene suture (Davis and Geck, Danbury, Conn.). The bypassed native circulation was ligated, diverting all flow through the two grafts. The retroperitoneum and abdomen were closed, wounds infiltrated with bupivacaine hydrochloride (Marcaine® 0.25%, Winthrop Pharm., New York, N.Y.) and the animals returned to single-animal cages until they were fully recovered from surgery. Perioperative analgesics included intramuscular ketorolac tromethamine (Toradol®, 30 mg load then 15 mg/8 hr, Syntex, Palo Alto, Calif.) and oral acetaminophen (Children's Tylenol®, 80 mg/6 hours, McNeil, Fort Washington, Pa.). Animals received 1.25 grains of aspirin 2 days before surgery and twice weekly thereafter. Animals were sedated weekly with ketamine (10 mg/kg i.m.) for duplex ultrasound interrogation of graft flow and for physical examination. The ultrasound examinations demonstrated no significant flow-limiting graft or anastomotic stenoses. Animals remained healthy and all grafts were patent throughout the period of study.

After 3 to 5 weeks animals were again anesthetized and grafts removed under general anesthesia at 3 weeks (2 animals), 4 weeks (one animal) and 5 weeks (one animal). All grafts were patent, providing one LNPoZ graft and one LPNSN graft from each animal. Animals were administered heparin and the grafts were excised and immediately rinsed with sterile saline and cut into 0.5 cm rings that were processed alternately into 10% buffered formalin, methyl Carnoy's fixative and OCT embedding media for frozen sections. For localization of β-Gal expressing smooth muscle cells, frozen sections were cut (0.5 μm thickness) from both β Gal (LNPoZ) and control (LPNSN) grafts and mounted onto glass slides. Sections were cut from analogous regions of the two grafts from an individual animal and were processed simultaneously. Slides were immersed in 0.5% glutaraldehyde for 10 minutes, washed 3 times in PBS, and sections were then covered with 100 μl of the X-Gal chromogen (5-bromo-4-chloro-3-indolyl-β-d-galactopyranoside; 5prime-3prime, Inc., Boulder, Colo.). Slides were then incubated 6 hrs at 37° C. in a moist chamber, rinsed in PBS, dehydrated in graded alcohols, coverslipped and examined by standard light microscopy for the presence of blue cytoplasmic staining to indicate β-gal expression.

To characterize graft cellular composition, paraffin embedded, methyl Carnoy's fixed sections from each graft were deparaffinized in xylene, rehydrated in graded alcohols and immunostained with antibodies specific for smooth muscle cell alpha-actin (1:500; Boehringer Mannheim), endothelial cell Factor-VIII related antigen (1:500, DAKO) and macrophage CD-68 (1:1000, DAKO). Primary antibodies were localized with appropriate biotinylated secondary antibodies and tertiary avidin-biotin-peroxidase staining (Vector Laboratories Inc., Burlingame, Calif.). Control slides were included using appropriate non-immune IgG as the primary antibody. Sections were counter-stained with methyl-green or hematoxylin and examined using standard light-microscopy. Adjacent sections were stained with hematoxylin and eosin for standard histology.

Gross inspection of grafts at the time of removal showed them to be well incorporated. The lumenal surface appeared to be incompletely endothelialized with islands of thin adherent thrombus. Both grafts had a similar appearance when stained with hematoxylin-eosin, and analysis of cross-sections by standard light microscopy showed a thin incompletely endothelialized neointima forming along the length of grafts. The graft wall was incorporated with microvessels and a cellular infiltrate. Both grafts also had a similar appearance when immunostained for smooth muscle cell alpha-actin, in that immunocytochemical staining demonstrated many alpha-actin positive cells. The brown alpha-actin staining identified smooth muscle cells throughout the graft wall, in the forming neointima as well as in clusters, in a pattern resembling the distribution of β-Gal positive cells, and in microvessels within the graft wall. Macrophages (CD68-positive cells) were seen largely within the PTFE graft wall with few seen in the forming neointima. This pattern is typical of healing porous PTFE grafts (Clowes et al., Am. J. Pathol. 123: 220-230 (1986)).

Histochemical staining with the X-Gal chromogen demonstrated clusters of vector-expressing cells within the graft wall interstitium of each β-Gal (LNPoZ) seeded graft. Many blue staining cells were seen localized in the wall of the graft seeded with the β-galactosidase expressing smooth muscle cells, in the mid and outer graft wall and not in the inner wall of the graft or the forming neointima, and in cross-section resembled the pattern observed with alpha actin staining. The control graft from the same animal, seeded with LPNSN-2-expressing cells, did not stain blue with X-Gal. The histologic appearance of the two grafts was otherwise indistinguishable. Vector expressing cells were not found within the fibrous tissue outside the graft wall. In addition, β-Gal expressing cells were not seen to localize in microvessels within the graft wall.

Prior to implantation, histologic cross-sections of cultured graft segments showed a fairly homogeneous pattern of cell seeding, although slightly more cells were present near the lumen of grafts before implantation. Thus, seeded smooth muscle cells survived better in the outer wall, or cells migrated to the outer wall preferentially after implantation. Grafts removed at 3, 4 or 5 weeks after implantation were similar in appearance, indicating that the events localizing smooth muscl cells to the mid and outer graft wall occurred early and the graft-cell organization then remained stable. Seeded smooth muscle cells remained in the wall of the graft and were not seen to contribute to the forming intima. Importantly, seeded smooth muscle cell were not found in connective tissue outside of the graft wall. This localization makes it possible to remove the entire population of transduced smooth muscle cells, if necessary, by removing the graft.

Although the foregoing invention has been described in some detail by way of illustration and example for purposes of clarity of understanding, it will be apparent that certain changes and modifications may be practiced within the scope of the appended claims.

What is claimed is:

1. A device for implanting autologous vascular smooth muscle cells transduced with a gene of interest in a patient, comprising:
a tubular elongate member having a wall, which wall has an interior surface, an exterior surface, and pores therein;
the autologous smooth muscle cells transduced with the gene of interest immobilized within the pores and upon the interior surface of the wall to form a tubular smooth muscle cell complex having an interior surface; and
autologous vascular endothelial cells adherent to the interior surface of the tubular smooth muscle cell complex.

2. A device as in claim 1, wherein the tubular elongate member is comprised of a porous synthetic material.

3. A device as in claim 2, wherein the porous synthetic material is polytetrafluoroethylene (PTFE), dacron or nylon.

4. A device as in claim 3, wherein the tubular elongate member is a vascular graft.

5. A device as in claim 1, wherein the autologous vascular smooth muscle cells are transduced with a gene encoding erythropoietin.

6. A device as in claim 1, wherein the vascular smooth muscle cells are transduced with a gene encoding granulocyte colony stimulating factor or granulocyte macrophage colony stimulating factor.

7. A device as in claim 1, wherein the vascular smooth muscle cells are transduced with a gene encoding Factor IX.

8. A device as in claim 1, wherein the transduced cells constitutively express an anticoagulant.

9. A device as in claim 1, wherein the transduced autologous vascular smooth muscle cells are immobilized to the tubular elongate member with a polymer.

10. A device as in claim 9, wherein the polymer is collagen or fibronectin.

11. A method for introducing a gene of interest to a patient comprising:
engrafting a device as in claim 1 into the patient's vascular system, wherein the transduced vascular smooth muscle cells contain the gene operably linked to a promoter for expression.

12. A method as in claim 11, wherein the gene encodes erythropoietin, granulocyte colony stimulating factor, granulocyte macrophage colony stimulating factor, or Factor IX.

13. A method as in claim 11, wherein the device is engrafted into the patient's arterial system.

14. A method for treating anemia in a patient, comprising engrafting a device as in claim 1 into the patient's vascular system, wherein the transduced autologous smooth muscle cells express erythropoietin.

15. The method of claim 14, wherein the device is engrafted into the patient's arterial system.

16. A method for treating an occlusion of a blood vessel in a patient, comprising engrafting a device as in claim 1 into the occluded blood vessel bypassing the occlusion, wherein the transduced cells constitutively express an anticoagulant protein.

17. A method as in claim 16, wherein the anticoagulant is a plasminogen activator or antithrombin-III.

18. A method as in claim 17, wherein the plasminogen activator is alteplase or urokinase.

19. A method for treating or preventing diabetes in a patient, comprising engrafting a device as in claim 1 into the patient, wherein the transduced cells constitutively express an insulin or proinsulin polypeptide.

20. A method for treating or preventing a disease in a mammal, comprising:
removing vascular endothelial cells and vascular smooth muscle cells from the mammal;
transducing the smooth muscle cells with a gene which encodes a product for treating or preventing the disease, operably linked to a promoter;
immobilizing on a tubular elongate porous vascular graft device the transduced smooth muscle cells within the pores and interior surface of the graft;
coating the interior of the graft device having immobilized thereon the transduced smooth muscle cells with the endothelial cells; and
engrafting the device having the immobilized transduced smooth muscle cells and endothelial cells into the vasculature of the mammal to treat or prevent the disease.

21. The method of claim 20, further comprising the step of cultivating the vascular smooth muscle cells obtained from the mammal in a medium containing autologous serum prior to immobilizing the cells on the vascular graft.

22. The method of claim 21, further comprising the step of cultivating the vascular endothelial cells obtained in a medium containing autologous serum prior to coating the vascular graft.
